(19) United States
(12) Patent Application Publication
(10) Pub. No.: US 2023/0135239 A1
(43) Pub. Date: May 4, 2023

(54) DRUG FOR TREATING SEVERE ACUTE RESPIRATORY SYNDROME CAUSED BY CORONAVIRUS AND COMPLICATIONS THEREOF AND USE OF THE DRUG

(71) Applicants: SHANGHAI PUBLIC HEALTH CLINICAL CENTER, Shanghai (CN); Shanghai Yizhe Biotechnology Co., Ltd., Shanghai (CN)

(72) Inventors: Wenqiang YU, Shanghai (CN); Zhenyan LI, Shanghai (CN); Jianqing XU, Shanghai (CN); Wei LI, Shanghai (CN); Cheng LIAN, Shanghai (CN)

(73) Assignees: SHANGHAI PUBLIC HEALTH CLINICAL CENTER, Shanghai (CN); Shanghai Yizhe Biotechnology Co., Ltd., Shanghai (CN)

(21) Appl. No.: 17/453,573

(22) Filed: Nov. 4, 2021

(51) Int. Cl. C12N 9/24; A61P 31/14; C07K 14/47; A61K 31/37

(52) U.S. Cl. CPC C12N 9/2402; A61P 31/14; C07K 14/4703; A61K 31/37

(57) ABSTRACT

The present invention discloses a drug for treating severe acute respiratory syndrome caused by coronavirus and the complications thereof and the use of the drug, and specifically the use of an agent in the preparation of a drug for treating severe acute respiratory syndrome caused by coronavirus and the complications thereof. The above-mentioned agent is at least one of hyaluronic acid synthesis inhibitor, hyaluronidase, hyaluronidase inhibitor, hyaluronic acid receptor inhibitor, and anticoagulant. The present invention is based on the discovery of an important pathogenic mechanism of severe acute respiratory syndrome-related coronavirus 2 that a sequence homologous to human in severe acute respiratory syndrome-related coronavirus 2, named HIS (Human Insert Sequence), can specifically activate hyaluronic acid synthase HAS and other inflammatory factors, and then it is found from clinical samples that the content of hyaluronic acid in the serum of a patient with severe COVID-19 is significantly higher than that in a patient with mild COVID-19. The above-mentioned agent is used for the first time for treating coronavirus and complications, and it is verified that a hyaluronic acid synthesis inhibitor can reduce the content of hyaluronic acid, thus the adjustment of hyaluronic acid level has good application value in the treatment and/or prevention of conditions caused by coronavirus.

### TECHNICAL FIELD

The present invention relates to the field of biotechnology, in particular to a drug for treating severe acute respiratory syndrome caused by coronavirus and complications thereof and use of the drug, specifically a method for treating severe acute respiratory syndrome caused by coronavirus and the complications thereof by adjusting the level of hyaluronic acid, and the use of an agent that can adjust the level of hyaluronic acid in the preparation of a drug for treating severe acute respiratory syndrome caused by coronavirus and the complications thereof.

### BACKGROUND OF THE INVENTION

Coronavirus is a type of unsegmented positive-sense single-stranded RNA virus with an envelope, can infect a variety of hosts such as mammals and birds, and can especially cause serious respiratory infections in human. In the past two decades, there have been two highly pathogenic coronaviruses that are zoonotic: severe acute respiratory syndrome-related coronavirus (SARS-CoV) and Middle East respiratory syndrome (MERS-CoV). Bats and camels are the natural hosts of SARS-CoV and MERS-CoV, respectively. Previously, six types of coronaviruses were known to infect human, comprising HCoV-229E, HCoV-NL63, HCoV-OC43, HCoV-HKU1, SARS-CoV and MERS-CoV.

Corona Virus Disease 2019, referred to as “COVID-19” briefly, refers to pneumonia caused by the infection of 2019 severe acute respiratory syndrome-related coronavirus 2. COVID-19 is an acute infectious pneumonia. Researchers have discovered that its pathogen is a new type of β-coronavirus that has not previously been found in human. The virus was subsequently named severe acute respiratory syndrome-related coronavirus 2 (SARS-CoV-2) by the World Health Organization (WHO). The initial symptoms of a patient with COVID-19 are mostly fever, fatigue and dry cough, and they gradually develop severe symptoms such as dyspnea. In some severe cases, acute respiratory distress syndrome (ARDS), septic shock and even death may occur. As of Jul. 7, 2020, SARS-CoV-2 has continued to spread in 188 countries and regions around the world, causing more than 11.62 million confirmed cases and 538,000 deaths. Currently, the molecular mechanism of SARS-CoV-2 causing COVID-19 is not clear, and effective specific drugs against SARS-CoV-2 are clinically absent. Therefore, there is an urgent need to develop a drug for treating COVID-19 caused by SARS-CoV-2.

Hyaluronic acid (HA) is a glycosaminoglycan with disaccharide (D-glucuronic acid and N-acetylglucosamine) as the basic unit, also known as uronic acid and hyaluronan. HA is synthesized by HA synthase (Hyaluronic Acid Synthase, HAS). HAS in vertebrate comprises HAS1, HAS2 and HAS3. Hyaluronidase is responsible for the degradation of HA, which can degrade high molecular weight HA into low molecular weight HA fragments.

HA is widely distributed in connective tissue, epithelial tissue and nerve tissue. HA can be divided into high molecular weight species or low molecular weight species, and the functions between the two species are different. The functions of HA and related molecules thereof mainly are that: (1) HA has the characteristics such as non-toxicity, low immune response and high biocompatibility, and is currently widely used in various eye surgery and skin care products; (2) HA plays a key role in the occurrence and development of tumors; the HA secreted by tumor cells can induce extracellular signal pathways by binding to the HA receptors (such as CD44) on the cell surface to promote the occurrence and development of tumors; hyaluronidase-mediated degradation of hyaluronic acid produces small molecular HA fragments, some of which (about 10-25 disaccharide units) have angiogenic activity. (3) Low molecular weight HA can mediate a variety of inflammatory reactions. In 1991, Waldenström et al. found that HA can increase local edema and promote the inflammatory cascade, leading to leukocyte migration, proliferation and differentiation. In 2005, Wellen et al. found that many chronic inflammatory diseases are related to HA deposition. For example, the pathogenesis of T2D comprises an inflammatory component and the inflammation is confined to muscle and adipose tissue, and HA deposition occurs frequently in muscle and adipose tissue.

4-MU (4-Methylumbelliferone) is a derivative of the coumarin family and a selective inhibitor for biosynthesis of hyaluronic acid. In terms of mechanism, the raw materials for HA synthesis are UDP glucuronic acid (UDP GlcUA) and UDP-N-acetylglucosamine (UDP GlcNAc), and these raw materials are respectively produced by transferring UDP residues to glucuronic acid and N-acetylglucosamine under the action of UDP glucuronyl transferase (UGT). 4-MU can be used as a competitive substrate for UGT, affecting the availability of UDP GlcUA and UDP GlcNAc, thereby inhibiting HA synthesis. 4-MU has been used in human, and Heparvit® (a health food for a patient with cancer) can be purchased without a prescription; in Europe and Asia, the prescription drug Hymecromone can be used to treat biliary tract spasms. The drug has excellent safety and has been used for several years. In addition, other coumarin derivatives, such as Marcumar® and Coumadin®, are mostly used as preventive drugs to reduce the occurrence of cardiovascular disease due to the anticoagulant mechanisms thereof.

In the early days, the inventors found the human-derived short conservative sequences are exist in the genome sequence of SARS-CoV-2 and are called human inserted sequences (HIS). It is found by analysis that the product of HIS can form a hairpin structure, and the product thereof is miRNA derived from virus. It is found that the adjacent HAS genes can be activated by transfecting cells with these HIS fragments containing human-derived conservative sequences, thus the expression of hyaluronic acid synthase is promoted and the level of HA is thereby up-regulated. In view of this, the inventors believe that the increase in the synthesis of hyaluronic acid is an important molecular mechanism for the SARS-COV-2 to cause COVID-19 and the complications thereof. Further, The HA content in the serum of a patient with severe COVID-19 is significantly higher than that in a patient with mild COVID-19. The up-regulation of HA level is closely related to the clinical symptoms of COVID-19, comprising pulmonary ground-glass lesions, lymphocyte decline (leukopenia), immune response and inflammatory factor storms, systemic vasculopathy, thrombosis and coagulopathy, and differences in the infectious ability of SARS-CoV-2 among different sexes. HA inhibitors can significantly down-regulate the concentration of HA and may improve the clinical symptoms of COVID-19. Therefore, regulating the synthesis and degradation pathways of hyaluronic acid is a new strategy for treating COVID-19. Similar HIS sequences also exist in SARS-CoV and MERS-COV. Therefore, inhibition and regulation of hyaluronic acid is a new strategy for treating severe acute respiratory syndrome caused by various coronaviruses and the complications thereof.

### SUMMARY OF THE INVENTION

In order to overcome the defect that effective specific drugs against coronavirus are absent in the prior art, the present invention provides a drug for treating severe acute respiratory syndrome caused by coronavirus and complications thereof and use of the drug.

In order to achieve the above objective, the following technical solutions are used in the present invention:

In the first aspect, the present invention provides the use of an agent in the preparation of a drug for treating severe acute respiratory syndrome caused by coronavirus and the complications thereof. The agent can inhibit the synthesis of hyaluronic acid, reduce the content of high molecular weight and/or low molecular weight hyaluronic acid, inhibit the decomposition of high molecular weight hyaluronic acid into low molecular weight hyaluronic acid, inhibit the binding of hyaluronic acid with a receptor of the hyaluronic acid or has an anticoagulant effect.

It is understandable that the agent in the claims of the present invention is any suitable substance with the exemplified functions, comprising a chemical substance, a biological substance and a mixture thereof, etc., and the substances not only used on the experimental scale but also used on the pharmaceutical product scale.

In order to further optimize the above-mentioned use, the technical measures used in the present invention also comprise:

further, the molecular weight of the high molecular weight hyaluronic acid is higher than 2000 KDa, and the molecular weight of the low molecular weight hyaluronic acid is 10 KDa-2000 KDa.

Further, the agent is at least one of hyaluronic acid synthesis inhibitor, hyaluronidase, hyaluronidase inhibitor, hyaluronic acid receptor inhibitor, and anticoagulant. Specifically, the hyaluronic acid synthesis inhibitor can be a selective inhibitor of hyaluronic acid biosynthesis, a hyaluronic acid synthase inhibitor, etc., and the hyaluronic acid receptor inhibitor is a competitive binder for hyaluronic acid receptor or a binding inhibitor for hyaluronic acid receptor, etc. The above-mentioned hyaluronidase is an enzyme that degrades hyaluronic acid, which can reduce the content of hyaluronic acid. Low molecular weight hyaluronic acid can cause inflammation and coagulopathy. Hyaluronidase can directly degrade low molecular weight hyaluronic acid, etc. The hyaluronidase inhibitor is a selective inhibitor of hyaluronidase, and can inhibit hyaluronidase activity and inhibit the degradation of high molecular weight hyaluronic acid into low molecular weight hyaluronic acid which causes inflammation.

Further, the hyaluronic acid synthesis inhibitor comprises at least one of hymecromone (also known as 4-Methylumbelliferone, for short 4-MU), Hitrechol, nicotinylmethylamide, hydroxymethylamine hydrochloride, oxymethylamine hydrochloride and hymecromone derivatives.

Further, in a specific embodiment, the hyaluronic acid synthesis inhibitor is hymecromone, and more preferably hymecromone tablet.

Further, the hyaluronic acid synthesis inhibitor has the ability to inhibit the synthesis of hyaluronic acid.

Further, the hyaluronic acid synthesis inhibitor has the ability to inhibit the increase of hyaluronic acid induced by pseudovirus.

Further, the hymecromone can reduce the mRNA levels of the hyaluronic acid synthase genes HAS1, HAS2 and HAS3.

Further, the hyaluronidase/HAase/Ronidase comprises at least one of hyaluronic acid-4-glycohydrolase (EC3.2.1.35), hyaluronic acid-3-glycohydrolase (EC3.2.1.36), hyaluronic acid lyase (EC4.2.2.1), hyaluronidase, hyaluronic acid glucosaminidase 1 (HYAL1), hyaluronic acid glucosaminidase 2 (HYAL2), hyaluronic acid glucosaminidase 3 (HYAL3), and hyaluronic acid glucosaminidase 4 (HYAL4). Further, the hyaluronidase has the ability to degrade hyaluronic acid, thereby accelerating the metabolism of hyaluronic acid, and eliminating conditions caused by high molecular weight hyaluronic acid during the pathogenesis.

Further, the hyaluronidase inhibitor comprises at least one of alkaloids, antioxidants, terpenoids, flavonoids, synthetic compounds, mucopolysaccharides, fatty acids, oligosaccharides, and anti-inflammatory drugs.

Further, the hyaluronidase inhibitor can block the process of hydrolyzing high molecular weight hyaluronic acid into low molecular weight hyaluronic acid, and eliminate conditions caused by small molecular hyaluronic acid during the pathogenesis.

Further, the hyaluronic acid receptors comprise CD44, TLR4, HABP2, RHAMM, LYVE-1, HARE and layilin.

Further, the hyaluronic acid receptor inhibitor inhibits the binding of hyaluronic acid to the receptor thereof by using a short peptide or a compound so as to block the hyaluronic acid receptor.

Further, CD44 receptor inhibitor comprises verbenalin, F-16438 A, B, E, F, G, etc.; HABP2 receptor inhibitor comprises TGF-β, Bikaverin, etc.; RHAMM receptor inhibitor comprises simvastatin, farnesylation, etc.; LYVE-1 receptor inhibitor comprises LYVE-1 neutralizing antibodies (mAb 2125 and C1/8), etc.; HARE inhibitor comprises neutralizing antibodies, etc.; Layilin inhibitor comprises neutralizing antibodies, etc.

Further, the anticoagulant comprises at least one of heparin, hirudin, Ca²⁺ chelators (sodium citrate, potassium fluoride), ethylenediaminetetraacetic acid (EDTA), sodium oxalate, sodium citrate, uPA inhibitor ZK824859, uPA inhibitor UK-371804, uPA inhibitor Amiloride, and uPAR inhibitor ATN615.

Further, the coronavirus comprises one of severe acute respiratory syndrome-related coronavirus 2 (SARS-CoV-2), severe acute respiratory syndrome-related coronavirus (SARS-CoV), and middle east respiratory syndrome coronavirus (MERS-CoV).

Further, the drug is a powder, a tablet, a granule, a capsule, a solution, an aerosol, an injection, an emulsion or a suspension.

Further, any suitable auxiliary material conventionally used can be used in the preparation process of the drug.

Further, the complications include but are not limited to: acute respiratory distress syndrome; vasculopathy; leukopenia; coagulation dysfunction; multiple organ failure, especially brain and testicular damage; sepsis; metabolic acidosis; respiratory failure; hypoxia in the body, etc.

In the second aspect, the present invention provides a method for treating severe acute respiratory syndrome caused by coronavirus and the complications thereof by adjusting the level of hyaluronic acid, comprising administering a drug in any suitable dosage form prepared from any of the above-mentioned agents for treatment.

Further, In the method, the applicable object is human or non-human mammals.

Still further, the non-human mammals comprise rodents (such as mice, rats, rabbits), and primates (such as monkeys).

Comparing with the prior art, the above technical solutions are used in the present invention, and the following technical effects are achieved:

Based on clinical samples, the present invention found that the content of hyaluronic acid in the serum of a patient with severe COVID-19 is significantly higher than that in a patient with mild COVID-19, the absolute number of lymphocytes in a patient with severe COVID-19 is significantly lower than that in a patient with mild COVID-19, and the content of D-dimer in a patient with severe COVID-19 is significantly higher than that in a patient with mild COVID-19. The hyaluronic acid (synthase) inhibitor, hyaluronidase, hyaluronidase inhibitor, hyaluronic acid receptor inhibitor or competitive binder, and anticoagulant are used for the first time for treating coronavirus and the complications thereof. In the present invention, the pseudoviruses are overexpressed in cell lines by means of the cytological experiments to simulate the infection process so as to specifically study the effect of hyaluronic acid synthesis inhibitors. The addition of a hyaluronic acid synthesis inhibitor can reduce the hyaluronic acid expressed by the cells overexpressing pseudovirus, thereby providing a theoretical basis for the use of the degradation of hyaluronic acid in the treatment of coronaviruses, and also verifying that the new strategy of the inhibition of hyaluronic acid to treat severe acute respiratory syndrome caused by various types of coronaviruses and the complications thereof is feasible and has good application value in the treatment and/or prevention of conditions caused by coronavirus.

### BRIEF DESCRIPTION OF THE DRAWINGS

FIG. 1 is a schematic diagram of the result of the mRNA level of the gene HAS1 after overexpression of the target fragments of coronavirus in 293T cells by qPCR detection in an embodiment of the present invention; wherein, **, p<0.01.***, p<0.001.

FIG. 2 is a schematic diagram of the result of the mRNA level of the gene HAS2 after overexpression of the target fragments of coronavirus in 293T cells by qPCR detection in an embodiment of the present invention; wherein, **, p<0.01.

FIG. 3 is a schematic diagram of the result of the mRNA level of the gene HAS3 after overexpression of the target fragments of coronavirus in 293T cells by qPCR detection in an embodiment of the present invention; wherein, **, p<0.01.***, p<0.001.

FIG. 4 is a comparison diagram of the content of hyaluronic acid in the serum of patients with mild or severe COVID-19 determined by ELISA in an embodiment of the present invention.

FIG. 5 is a comparison diagram of the absolute number of lymphocytes in the serum of patients with mild or severe COVID-19 determined by ELISA in an embodiment of the present invention.

FIG. 6 is a comparison diagram of the D-dimer content in the serum of patients with mild or severe COVID-19 determined by ELISA in an embodiment of the present invention.

FIG. 7 is a comparison diagram of the content of hyaluronic acid in cell culture solution after overexpression of pseudovirus in 293T and MRCS and addition of hyaluronic acid synthesis inhibitor as determined by ELISA in an embodiment of the present invention; wherein, part A is the content of hyaluronic acid in cell culture solution after addition of hyaluronic acid synthesis inhibitor to 293T, SARS is SARS-CoV, SARS-2 is a fragment of SARS-CoV-2, and SARS-2* is the other fragment of SARS-CoV-2, *: p<0.05, **: p<0.01; wherein, part B is the content of hyaluronic acid in cell culture solution after addition of hyaluronic acid synthesis inhibitor to MRCS, SARS is SARS-CoV, SARS-2 is a fragment of SARS-CoV-2, and SARS-2* is the other fragment of SARS-CoV-2, *: p<0.05.

FIG. 8 is a schematic diagram of the results of quantification of the mRNA of HAS1, HAS and HAS3 after overexpression of pseudovirus in 293T and addition of hymecromone in an embodiment of the present invention; wherein, SARS is SARS-CoV, SARS-2 is a fragment of SARS-CoV-2, and SARS-2* is the other fragment of SARS-CoV-2, *: p<0.05, **: p<0.01, ***: p<0.001.

FIG. 9 is a comparison diagram of the changes in the content of hyaluronic acid in the cell culture solution after overexpression of pseudovirus in 293T and addition of hymecromone as determined by ELISA in an embodiment of the present invention; wherein, SARS is SARS-CoV, SARS-2 is a fragment of SARS-CoV-2, and SARS-2* is the other fragment of SARS-CoV-2.

FIG. 10 is a schematic diagram of the results of quantification of the mRNA of HAS1, HAS2 and HAS3 after overexpression of pseudovirus in 293T and addition of gradient hymecromone in an embodiment of the present invention.

FIG. 11 is a comparison diagram of the changes in the content of hyaluronic acid in the cell culture solution after overexpression of pseudovirus in 293T and addition of gradient hymecromone as determined by ELISA in an embodiment of the present invention.

### DETAILED DESCRIPTION OF THE INVENTION

The specific implementations of the present invention will be further described below in conjunction with the drawings and examples. The following examples are only used to illustrate the technical solutions of the present invention more clearly, and cannot be used to limit the scope of protection of the present invention. In experimental methods in the following examples where no specific conditions are indicated, choices can be made according to conventional methods and conditions in the art or commodity instructions. In the following examples, the agents whose sources are not indicated are all substances conventionally used in the art or can be prepared by conventional technical means. In the following examples, each of the short conservative sequence HIS can be obtained by conventional Blast alignment, and the related primer sequences can be obtained by conventional design through software such as Primer 5.0. In the following examples, the pseudoviruses constructed comprise SARS-CoV-2, SARS-CoV, and MERS-COV. The overexpression vectors of the above-mentioned pseudoviruses are all prepared by conventional techniques in the art. The molecular cloning technology in the following examples provides a method for purifying and amplifying specific DNA fragments at the molecular level in the prior art.

### EXAMPLE 1

### The Effect of Overexpression of the Target Sequence of the RNA Virus in Cells on the Expression Level of Surrounding Genes

In this example, the effect of the overexpression of the target sequences of the RNA virus in 293T cells on the expression level of surrounding genes was detected. The steps are briefly described as follows:

1. Preparation of lentivirus by liposome method: According to molecular cloning, SARS-CoV-2, SARS-CoV, MERS-CoV overexpression plasmid, virus packaging plasmid psPAX2 and capsid plasmid pMD2.G-VSVG were transferred into 293T cells, and the supernatant was collected after 48 hr and 72 hr, respectively. The cell debris was filtered through a 0.45 μm filter to obtain the lentivirus stock solution.

2. Cell infection: 200,000 cells to be infected (lentiviral stock solution) was spread in a 6 cm culture dish in advance, after the cells adhered on the second day, the first infection was carried out, and the infection was repeated again on the third day; on the fourth day, the cells were allowed to recover for one day without adding any stimulation; on the fifth day, drug screening was started to perform based on corresponding markers carried by the plasmid that reduce the potency of the drug.

3. Real-time fluorescence quantitative PCR

(1) Total RNA Extraction 10⁶-10⁷ cells were prepared, resuspended in PBS, and then centrifuged to remove the supernatant, 1 ml of Trizol was added for lysis at room temperature for 5 min, then 0.2 ml of chloroform was added. The mixture was shaken in a vortex shaker for 15 s, and left to stand at room temperature for 2 min. The mixture was centrifuged in a centrifuge at 4° C. for 15 min at 13,300 rpm. The upper colorless water phase was transferred into another EP tube. An equal volume of isopropanol was added, mixed thoroughly in a vortex shaker, and the mixture was centrifuged in a centrifuge at 4° C. at 13,300 rpm for 10 min. The supernatant was discarded, and 1 ml of 75% ethanol prepared with DEPC water was added, turned upside down until the precipitate was suspended, and centrifuged in a centrifuge at 4° C. at 13,300 rpm for 5 min. The supernatant was aspirated with a pipette, during the period of drying at room temperature for 5-20 min, the morphology of the precipitation was observed. When just being transparent, 40-100 μl of DEPC water was used for dissolution according to the amount of precipitation. 1 μl was taken and the concentration and OD260/OD280 was measured on Nanodrop. The extracted RNA was stored in a refrigerator at −80° C.

(2) Reverse Transcription Synthesis of cDNA

Takara (D2680A) reverse transcription PCR kit was used, the PCR reaction system and program were as follows:

| Reverse transcription PCR System | Total volume 20 μl | |
|---|---|---|
| 5× PrimeScript Buffer | 4 | μl |
| dNTP Mixture (2.5 mM each) | 4 | μl |
| Random 6 mers (100 μM) | 1 | μl |
| Oligo dT Primer (50 μM) | 1 | μl |
| PrimeScript Reverse Transcriptase (200 U/μl) | 0.5 | μl |
| RNase Inhibitor (40 U/μl) | 0.5 | μl |
| Total RNA | 1 | μg |

| | |
|---|---|
| RNase Freed H2O | up to 20 μl |

Reverse transcription PCR program: 42° C. 10 min, 95° C. 2 min.

(3) RT-qPCR

The expression of the gene of interest at the transcription level was detected using Takara real-time fluorescent quantitative PCR kit.

| Real-time fluorescence | |
|---|---|
| quantitative PCR System | Total volume 10 μl |
| Sybr Green Mix | 5 μl |
| Forward (10 μm) | 1 μl |
| Reverse (10 μm) | 1 μl |
| cDNA | 3 μl |

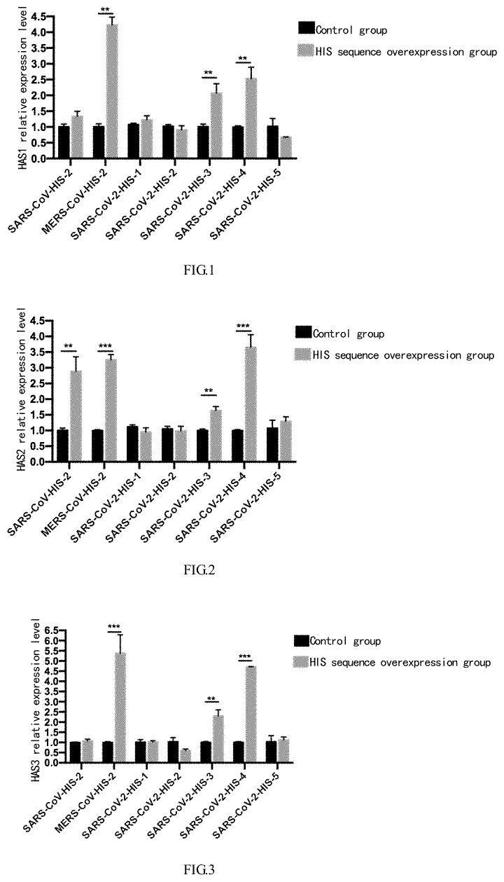

Experimental results: After the overexpression of the target sequence fragment, the HAS1 (FIG. 1), HAS2 (FIG. 2) and HAS3 (FIG. 3) genes of the hyaluronic acid synthase family related to severe acute respiratory syndrome-related coronavirus 2 were also significantly activated by SARS-CoV-HIS, MERS-CoV-HIS, SARS-COV-2-HIS-3 and SARS-COV-2-HIS-4 fragments.

### EXAMPLE 2

### Concentration of Hyaluronic Acid HA in the Serum as Determined by ELISA

In this example, the content of hyaluronic acid in the serum of patients with mild or severe COVID-19 was determined by ELISA. The experiment was carried out in accordance with the instructions of the HA ELISA kit (R & D, DY3614-05) from R & D. The steps are briefly described as follows:

1. Coating ELISA plate: The plate was coated with 100 μl/well of Capture Reagent overnight.

2. Sealing: The Capture Reagent was removed by patting the plate. The plate was washed 3 times with 400 μl/well of Wash buffer and patted to dryness. The plate was sealed with 100 μl/well of Dilute Reagent for 1 h.

3. Washing the plate and incubating the sample: The plate was washed with 400 μl/well of Wash buffer 3 times, 100 μl/well of standard and serum to be tested were added (100 μl of the serum from patients with mild and severe COVID-19 was diluted with 200 μl of Dilute Reagent in the kit to a total volume of 300 μl, 3 replicate wells were made), and incubated at room temperature for 2 h.

4. Washing the plate and incubation with the Detect Reagent. The plate was washed with 400 μl/well of Wash buffer 3 times, 100 μl/well of Detect Reagent was added and incubated at room temperature for 2 h.

5. Washing the plate and incubation with HRP. The plate was washed with 400 μl/well of Wash buffer 3 times, 100 μl/well of HRP was added and incubated at room temperature for 20 min.

6. Washing the plate and incubation with the substrate. The plate was washed with 400 μl/well of Wash buffer 3 times, 100 μl/well of mixed solution of substrates A and B was added and incubated at room temperature for 20 min.

7. Stopping color development. 50 μl/well of stop solution was added. Absorbance was read at 450 nm within 15 min.

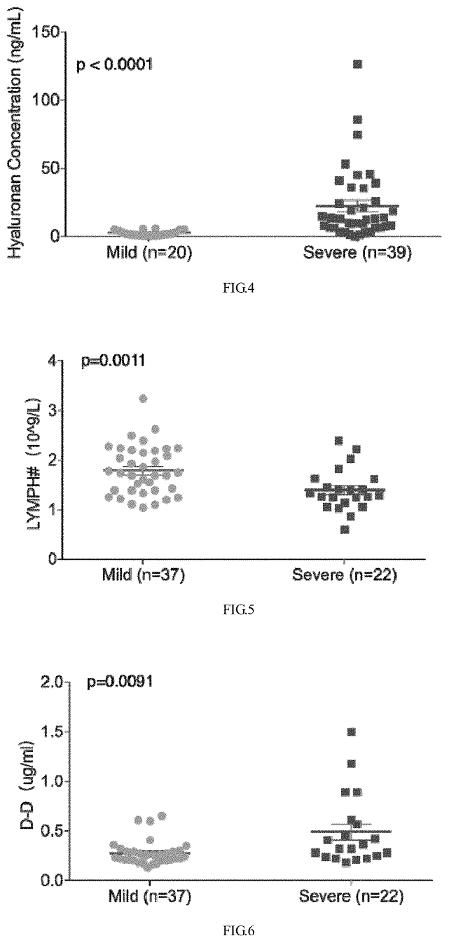

The results of the above detection for the HA content in the serum are shown in FIG. 4. The HA content in the serum of a patient with severe COVID-19 was significantly increased compared with that in a patient with mild COVID-19.

### EXAMPLE 3

### Detection of Blood Routine Indexes

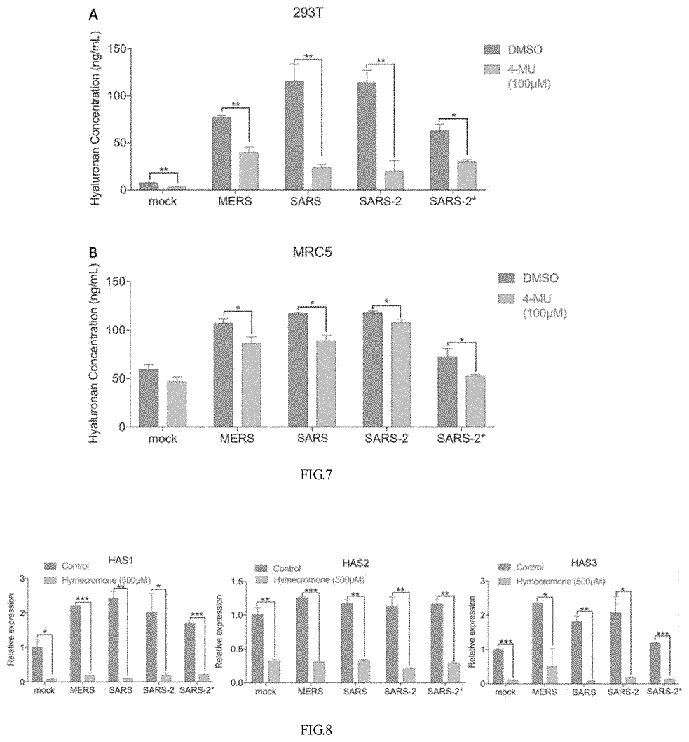

In this example, the absolute number of lymphocytes and the D-dimer content in the serum of patients with mild or severe COVID-19 were determined by ELISA. The blood routine indexes were provided by the hospital, wherein the number of lymphocytes in a patient with severe COVID-19 was significantly lower than that in a patient with mild COVID-19, suggesting that the number of the immune cells in a patient was decreased with the disease progressing to severe (as shown in FIG. 5); furthermore, D-dimer is a fibrin degradation product, and the increase of D-dimer level indicates the existence of hypercoagulable state and secondary hyperfibrinolysis in the body. Therefore, the mass concentration of D-dimer has diagnostic significance for thrombotic diseases. The content of D-dimer in the serum of a patient with severe COVID-19 was significantly higher than that in a patient with mild COVID-19, indicating that the risk of coagulation in a patient was increased with the condition of COVID-19 progressing to severe, and also indicating that there was a certain feasibility of subsequent anticoagulation therapy (as shown in FIG. 6).

### EXAMPLE 4

### Hyaluronic Acid Synthesis Inhibitor 4-MU Reduces Hyaluronic Acid Produced by Coronavirus Pseudovirus

In this example, the content of hyaluronic acid in cell culture solution after overexpression of pseudoviruses in 293T and MRCS and addition of hyaluronic acid synthesis inhibitor was determined by ELISA. The steps are briefly described as follows:

1. Preparation of lentivirus by liposome method: According to molecular cloning, SARS-CoV-2, SARS-CoV, MERS-CoV overexpression plasmid, virus packaging plasmid psPAX2 and capsid plasmid pMD2.G-VSVG were transferred into 293T and MRCS cells, and the supernatant was collected after 48 hr and 72 hr, respectively. The cell debris was filtered through a 0.45 μm filter to obtain the virus stock solution.

2. Cell infection: 200,000 293T and MRCS cells to be infected was spread in a 6 cm culture dish in advance, after the cells adhered on the second day, the first infection was carried out, and the infection was repeated again on the third day; on the fourth day, the cells were allowed to recover for one day without adding any stimulation; on the fifth day, drug screening was started to perform based on corresponding markers carried by the plasmid that reduce the potency of the drug.

3. Replacement with the fresh medium was performed, 100 μM of hyaluronic acid synthesis inhibitor 4-MU was added in the experimental group, and DMSO (the solvent for 4-MU) was added in the control group. After 24 hours, the cell supernatant was collected and detected with hyaluronic acid ELISA kit (R & D, DY3614-05).

The detection results of the content of hyaluronic acid are shown in FIG. 7. The content of hyaluronic acid in the cell supernatant was significantly increased in the pseudovirus group, and the hyaluronic acid synthesis inhibitor can significantly reduce the increase in the content of hyaluronic acid caused by the pseudovirus, Consistent results were obtained in the cell lines 293T (part A of FIG. 7) and MRCS (part B of FIG. 7).

### EXAMPLE 5

### The Hyaluronic Acid Synthesis Inhibitor, Hymecromone Tablet Reduces the mRNA Levels of the Hyaluronic Acid Synthase Genes HAS1, HAS2 and HAS3

In this example, the mRNAs of HAS1, HAS2 and HAS3 were quantified after overexpression of pseudovirus in 293T and addition of hymecromone. The operation steps are briefly described as follows:

1. Preparation of lentivirus by liposome method: According to molecular cloning, SARS-CoV-2, SARS-CoV, MERS-CoV overexpression plasmid, virus packaging plasmid psPAX2 and capsid plasmid pMD2.G-VSVG were transferred into 293T cells, and the supernatant was collected after 48 hr and 72 hr, respectively. The cell debris was filtered through a 0.45 μm filter to obtain the virus stock solution.

2. Cell infection: 200,000 cells to be infected was spread in a 6 cm culture dish in advance, after the cells adhered on the second day, the first infection was carried out, and the infection was repeated again on the third day; on the fourth day, the cells were allowed to recover for one day without adding any stimulation; on the fifth day, drug screening was started to perform based on corresponding markers carried by the plasmid that reduce the potency of the drug.

3. Replacement with the fresh medium was performed, 500 μM of hyaluronic acid synthesis inhibitor tablet, hymecromone tablet was added in the experimental group, and DMSO (the solvent for hymecromone tablet) was added in the control group. The cells were collected after 48 hr.

4. Real-time fluorescence quantitative PCR

(1) Total RNA Extraction

10⁶-10⁷ cells were prepared, resuspended in PBS, and then centrifuged to remove the supernatant, 1 ml of Trizol was added for lysis at room temperature for 5 min, then 0.2 ml of chloroform was added. The mixture was shaken in a vortex shaker for 15 s, and left to stand at room temperature for 2 min. The mixture was centrifuged in a centrifuge at 4° C. for 15 min at 13,300 rpm. The upper colorless water phase was transferred into another EP tube. An equal volume of isopropanol was added, mixed thoroughly in a vortex shaker, and the mixture was centrifuged in a centrifuge at 4° C. at 13,300 rpm for 10 min. The supernatant was discarded, and 1 ml of 75% ethanol prepared with DEPC water was added, turned upside down until the precipitate was suspended, and centrifuged in a centrifuge at 4° C. at 13,300 rpm for 5 min. The supernatant was aspirated with a pipette, during the period of drying at room temperature for 5-20 min, the morphology of the precipitation was observed. When just being transparent, 40-100 μl of DEPC water was used for dissolution according to the amount of precipitation. 1 μl was taken and the concentration and OD260/OD280 was measured on Nanodrop. The extracted RNA was stored in a refrigerator at −80° C.

(2) Reverse Transcription Synthesis of cDNA

Takara (D2680A) reverse transcription PCR kit was used, the PCR reaction system and program were as follows:

| Reverse transcription PCR System | Total volume 20 μl | |
|---|---|---|
| 5× PrimeScript Buffer | 4 | μl |
| dNTP Mixture (2.5 mM each) | 4 | μl |
| Random 6 mers (100 μM) | 1 | μl |
| Oligo dT Primer (50 μM) | 1 | μl |
| PrimeScript Reverse Transcriptase (200 U/μl) | 0.5 | μl |
| RNase Inhibitor (40 U/μl) | 0.5 | μl |
| Total RNA | 1 | μg |

| | |
|---|---|
| RNase Free dH₂O | up to 20 μl |

Reverse transcription PCR program: 42° C. 10 min; 95° C. 2 min.

(3) RT-qPCR

The expression of the gene of interest at the transcription level was detected using Takara real-time fluorescent quantitative PCR kit.

| Real-time fluorescence | |
|---|---|
| quantitative PCR System | Total volume 10 μl |
| Sybr Green Mix | 5 μl |
| Forward (10 μm) | 1 μl |
| Reverse (10 μm) | 1 μl |
| DNA | 3 μl |

The mRNA levels of the hyaluronic acid synthase genes HAS1, HAS2 and HAS3 are shown in FIG. 8, and the hymecromone tablet can significantly reduce the mRNA levels of the hyaluronic acid synthase genes HAS1, HAS2 and HAS3.

### EXAMPLE 6

### Hyaluronic Acid Synthesis Inhibitor Tablet, Hymecromone Tablet Reduces Hyaluronic Acid Produced by Coronavirus Pseudovirus

In this example, the content of hyaluronic acid in the cell culture solution after addition of hyaluronic acid synthesis inhibitor tablet, hymecromone tablet to 293T was determined by ELISA. The operation steps are briefly described as follows:

1. Preparation of lentivirus by liposome method: According to molecular cloning, SARS-CoV-2, SARS-CoV, MERS-CoV overexpression plasmid, virus packaging plasmid psPAX2 and capsid plasmid pMD2.G-VSVG were transferred into 293T cells, and the supernatant was collected after 48 hr and 72 hr, respectively. The cell debris was filtered through a 0.45 μm filter to obtain the virus stock solution.

2. Cell infection: 200,000 cells to be infected was spread in a 6 cm culture dish in advance, after the cells adhered on the second day, the first infection was carried out, and the infection was repeated again on the third day; on the fourth day, the cells were allowed to recover for one day without adding any stimulation; on the fifth day, drug screening was started to perform based on corresponding markers carried by the plasmid that reduce the potency of the drug.

3. Replacement with the fresh medium was performed, 500 μM of hyaluronic acid synthesis inhibitor tablet, hymecromone tablet was added in the experimental group, and DMSO (the solvent for hymecromone tablet) was added in the control group. After 24 hours, the cell supernatant was collected and detected with hyaluronic acid ELISA kit (R & D, DY3614-05).

The detection results of the content of hyaluronic acid are shown in FIG. 9. The content of hyaluronic acid in the cell supernatant was significantly increased in the pseudovirus group, and the hyaluronic acid synthesis inhibitor can significantly reduce the increase in the content of hyaluronic acid caused by the pseudovirus.

### EXAMPLE 7

### The Effect of Different Concentration Gradients of Hymecromone Tablet on the mRNA Levels of Hyaluronic Acid Synthase Genes HAS1, HAS2 and HAS3

In this example, the mRNAs of HAS1, HAS2 and HAS3 were quantified after overexpression of pseudovirus in 293T and addition of hymecromone. The operation steps are briefly described as follows:

1. 200,000 293T cells (overexpressing SARS-CoV) were spread in a 6 cm culture dish, after 24 hours, replacement with the fresh medium was performed, 100 μM, 500 μM and 1000 μM of hyaluronic acid synthesis inhibitor tablet, hymecromone tablet were added in the experimental group, respectively, and DMSO (the solvent for hymecromone tablet) was added in the control group. The cells were collected after 48 hr.

2. The expression of the gene of interest at the transcription level was detected using the same real-time fluorescent quantitative PCR steps as that in example 5.

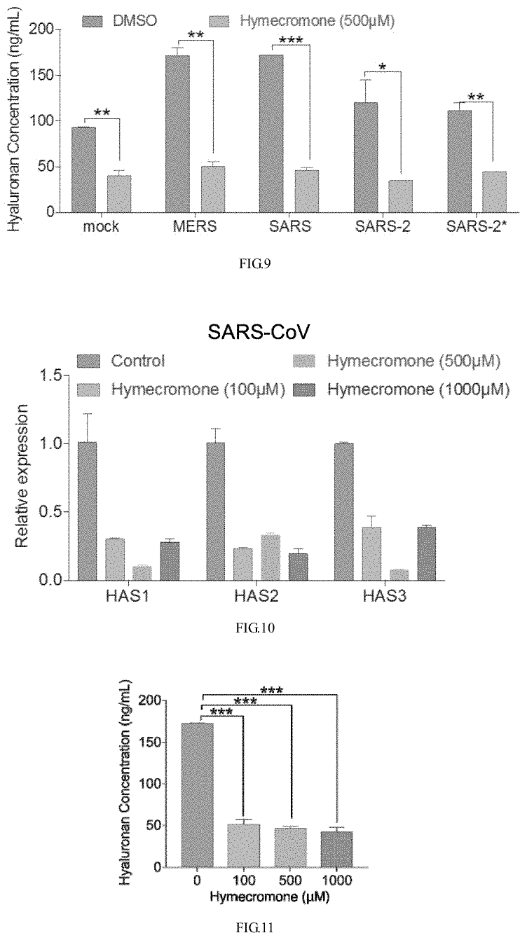

The mRNA levels of the hyaluronic acid synthase genes HAS1, HAS2 and HAS3 are shown in FIG. 10, and 100 μM of the hymecromone tablet can significantly reduce the mRNA levels of the hyaluronic acid synthase genes HAS1, HAS2 and HAS3.

### EXAMPLE 8

### Effect of Gradient Concentration of Hyaluronic Acid Synthesis Inhibitor Tablet, Hymecromone Tablet on the Content of Hyaluronic Acid

In this example, the content of hyaluronic acid in cell culture solution after overexpression of pseudoviruses in 293T and addition of gradient hymecromone was determined by ELISA. The steps are briefly described as follows:

1. Preparation of lentivirus of severe acute respiratory syndrome-related coronavirus (SARS-CoV) pseudovirus by liposome method: According to molecular cloning, SARS-CoV overexpression plasmid, virus packaging plasmid psPAX2 and capsid plasmid pMD2.G-VSVG were transferred into 293T cells, and the supernatant was collected after 48 hr and 72 hr, respectively. The cell debris was filtered through a 0.45 μm filter to obtain the virus stock solution.

2. Cell infection: 200,000 cells to be infected was spread in a 6 cm culture dish in advance, after the cells adhered on the second day, the first infection was carried out, and the infection was repeated again on the third day; on the fourth day, the cells were allowed to recover for one day without adding any stimulation; on the fifth day, drug screening was started to perform based on corresponding markers carried by the plasmid that reduce the potency of the drug.

3. Replacement with the fresh medium was performed, 100 μM, 500 μM and 1000 μM of hyaluronic acid synthesis inhibitor tablet, hymecromone tablet were added in the experimental group, respectively, and DMSO (the solvent for hymecromone tablet) was added in the control group. After 24 hours, the cell supernatant was collected and detected with hyaluronic acid ELISA kit (R & D, DY3614-05).

The detection results of the content of hyaluronic acid are shown in FIG. 11. The 100 μM, 500 μM and 1000 μM of hymecromone tablet can all inhibit the content of hyaluronic acid, and the hymecromone tablet at concentration of 100 μM can achieve the same inhibition effect.

It can be seen from the above examples that in the samples of patients with COVID-19, the content of hyaluronic acid in a patient with severe COVID-19 is significantly higher than that in a patient with mild COVID-19, the absolute number of lymphocytes in a patient with severe COVID-19 is significantly lower than that in a patient with mild COVID-19, and the content of D-dimer in a patient with severe COVID-19 is significantly higher than that in a patient with mild COVID-19. Moreover, the use of 100 μM of hyaluronic acid synthesis inhibitor 4-MU or hymecromone tablet can reduce the expression of hyaluronic acid in cells infected with pseudovirus, which provides a new strategy that the degradation of hyaluronic acid can be used to treat coronaviruses.

The specific examples of the present invention are described in detail above and are only for illustration, and the present invention is not limited to the specific examples described above. For a person skilled in the art, any equivalent modifications and alternatives made to the present invention are also within the scope of the present invention. Therefore, all equivalent changes and modifications made without departing from the spirit and scope of the present invention should fall within the scope of the present invention.

1. A method of treating severe acute respiratory syndrome caused by coronavirus and the complications of the severe acute respiratory syndrome caused by coronavirus, comprising administering an agent that inhibits synthesis of hyaluronic acid, reduces the content of high molecular weight and/or low molecular weight hyaluronic acid, inhibits the decomposition of high molecular weight hyaluronic acid into low molecular weight hyaluronic acid, inhibits the binding of hyaluronic acid with a receptor of the hyaluronic acid or has an anticoagulant effect.

2. The method according to claim 1, wherein the molecular weight of the high molecular weight hyaluronic acid is higher than 2000 KDa, and the molecular weight of the low molecular weight hyaluronic acid is 10 KDa-2000 KDa.

3. The method according to claim 1, wherein the agent is at least one of a hyaluronic acid synthesis inhibitor, a hyaluronidase, a hyaluronidase inhibitor, a hyaluronic acid receptor inhibitor, or an anticoagulant.

4. The method according to claim 3, wherein the hyaluronic acid synthesis inhibitor comprises at least one of hymecromone, Hitrechol, nicotinylmethylamide, hydroxymethylamine hydrochloride, oxymethylamine hydrochloride or hymecromone derivatives.

5. The method according to claim 4, wherein the hyaluronic acid synthesis inhibitor is hymecromone.

6. The method according to claim 4, wherein the hymecromone reduce the mRNA levels of the hyaluronic acid synthase genes HAS1, HAS2 and HAS3.

7. The method according to claim 3, wherein the hyaluronidase comprises at least one of hyaluronic acid-4-glycohydrolase, hyaluronic acid-3-glycohydrolase, hyaluronic acid lyase, hyaluronidase, hyaluronic acid glucosaminidase 1, hyaluronic acid glucosaminidase 2, hyaluronic acid glucosaminidase 3, and hyaluronic acid glucosaminidase 4.

8. The method according to claim 3, wherein the hyaluronidase inhibitor comprises at least one of alkaloids, antioxidants, terpenoids, flavonoids, synthetic compounds, mucopolysaccharides, fatty acids, oligosaccharides, and anti-inflammatory drugs.

9. The method according to claim 3, wherein the hyaluronic acid receptor inhibitor inhibits the binding of hyaluronic acid to a receptor of the hyaluronic acid by using a short peptide or a compound; wherein, the receptor of the hyaluronic acid comprises CD44, TLR4, HABP2, RHAMM, LYVE-1, HARE and layilin.

10. The method according to claim 3, wherein the anticoagulant comprises at least one of heparin, hirudin, Ca²⁺ chelators, ethylenediaminetetraacetic acid, sodium oxalate, sodium citrate, uPA inhibitors, and uPAR inhibitors.

11. The method according to claim 1, wherein the coronavirus comprises one of severe acute respiratory syndrome-related coronavirus 2 (SARS-CoV-2), severe acute respiratory syndrome-related coronavirus (SARS-CoV), and middle east respiratory syndrome coronavirus (MERS-CoV).

12. The method according to claim 1, wherein the agent is a powder, a tablet, a granule, a capsule, a solution, an aerosol, an injection, an emulsion or a suspension.

13. The method according to claim 1, wherein the complications comprise acute respiratory distress syndrome, vasculopathy, leukopenia, coagulation dysfunction, multiple organ failure, sepsis, metabolic acidosis, respiratory failure, and hypoxia in the body.
